(19) United States
(12) Patent Application Publication
(10) Pub. No.: US 2023/0212178 A1
(43) Pub. Date: Jul. 6, 2023

(54) METHOD OF PRODUCING PHOTOREACTIVE NUCLEOTIDE ANALOG

(71) Applicant: JAPAN ADVANCED INSTITUTE OF SCIENCE AND TECHNOLOGY, Nomi-shi, Ishikawa (JP)

(72) Inventor: Kenzo FUJIMOTO, Ishikawa (JP)

(73) Assignee: JAPAN ADVANCED INSTITUTE OF SCIENCE AND TECHNOLOGY, Nomi-shi, Ishikawa (JP)

(21) Appl. No.: 17/427,376

(22) PCT Filed: Jan. 27, 2020
(86) PCT No.: PCT/JP2020/002851

§ 371 Date: Jul. 30, 2021

(30) Foreign Application Priority Data
Jan. 30, 2019 (JP) 2019-014890

(51) Int. Cl. C07D 491/052; C07H 19/24; C07H 21/04

(52) U.S. Cl. CPC C07D 491/052; C07H 19/24; C07H 21/04

(57) ABSTRACT

In the present invention, a method for producing a compound of formula I comprises a step for causing a compound of formula III to undergo a Pechmann condensation reaction with respect to a compound of formula II in the presence of an organic solvent and an acid catalyst to obtain a compound of formula IV, and due to such method, provided are a novel photoreactive compound and a method for producing same that can be used for nucleic acid photoreaction technology.

### FIELD OF THE INVENTION

A sequence listing in electronic (ASCII text file) format is filed with this application and incorporated herein by reference. The name of the ASCII text file is “P-2244-WO-ST25-rev2.txt”; the file was created on Apr. 1, 2022; the size of the file is 821 bytes.

The present invention relates to a method of producing a photoresponsive nucleotide analog that can be photocrosslinked by light in visible light region.

### BACKGROUND OF THE INVENTION

Basic techniques in the field of molecular biology include ligation of nucleic acids and crosslinking of nucleic acids. The ligation and crosslinking of nucleic acids are used for introduction of genes or detection of nucleotide sequences, or inhibition of gene expressions, for example, in combination with hybridization. Therefore, the techniques of the ligation and crosslinking of nucleic acids are very important techniques that are used in basic molecular biology researches, as well as, for example, diagnosis or treatment in the medical field, or development or production of therapeutic agents and diagnostic agents, or development or production of enzymes, microorganisms or the like in the industrial and agricultural fields.

Known as photoreaction techniques of nucleic acids are photoligation techniques using 5-cyanovinyldeoxyuridine (Patent Literature 1: Japanese Patent No. 3753938 B; Patent Literature 2: Japanese Patent No. 3753942 B); and photocrosslinking techniques using modified nucleosides having a 3-vinylcarbazole structure at the base site (Patent Literature 3: Japanese Patent No. 4814904 B; Patent Literature 4: Japanese Patent No. 4940311 B).

### CITATION LIST

### Patent Literatures

[Patent Literature 1] Japanese Patent No. 3753938 B
[Patent Literature 2] Japanese Patent No. 3753942 B
[Patent Literature 3] Japanese Patent No. 4814904 B
[Patent Literature 4] Japanese Patent No. 4940311 B

### SUMMARY OF THE INVENTION

### Technical Problem

Because of the importance of the photoreaction technique of nucleic acids, there is a further need for novel compounds that can be used for the photoreaction technique of nucleic acids. An object of the present invention is to provide a novel photoreactive compound that can be used for a photoreaction technique of nucleic acids, and a method of producing the same.

### Solution to Problem

As a result of intensive studies for photoreactive compound that will be photoreactive crosslinking agent capable of being used for the photoreaction technique of nucleic acids, the present inventors have found that a compound having a pyranocarbazole skeleton structure in place of a base moiety of a nucleic acid will be such a photoreactive crosslinking agent capable of being used for the photoreaction technique of nucleic acids.

The compound has a characteristic pyranocarbazole structure and exhibits a photocrosslinking property due to such a relatively small structure. Therefore, the compound can be variously modified and used in various applications. Furthermore, the characteristic structure of the compound is similar to a base of nucleic acid. Therefore, the compound can be used as an artificial base (artificial nucleic acid base). That is, the characteristic structure of the compound can be introduced as an artificial base to produce an artificial nucleoside (a nucleoside analog) and an artificial nucleotide (a nucleotide analog), and also an artificial nucleic acid (a modified nucleic acid) containing such an artificial nucleotide. When such an artificial nucleic acid forms a crosslink by photoreaction, it will form a photocrosslink formed from one strand to other strand of a double helix. Therefore, the photoreactive nucleic acids can be used as double helix photo-crosslinkers capable of reaction that is specific to a desired sequence.

A photoreactive crosslinking agent using the compound has a feature capable of being photocrosslinked by irradiation with light having a wavelength longer than that of the conventional one, for example, irradiation with light in the visible light region, which feature is derived from the characteristic pyranocarbazole structure. Therefore, when it is desired to avoid any damage to DNAs and cells as much as possible, the photoreactive crosslinking agent is particularly advantageous because it can be photocrosslinked by irradiation with light having a long wavelength.

It should be noted that the photoreactive compound initiates a photoreaction by light irradiation, but the term “photoreactive” may be referred to as “photoresponsive” for emphasizing the meaning that a compound which has previously been stable initiates reaction in response to a signal of the light irradiation.

The present inventors have further researched the compound having the pyranocarbazole skeleton structure, and found that the addition of a substituent to a specific position of the pyranocarbazole skeleton structure has resulted in a compound which maintains excellent photoreactivity, and which can be very efficiently synthesized by a method as described later, and they have arrived at the present invention. Further, according to this production method, a photoreactive compound having a pyranocarbazole skeleton structure can be synthesized in a short period of time and with a higher yield.

Therefore, the present invention includes the following aspects (1) to (7): (1)

A method for producing a compound of the following formula I:

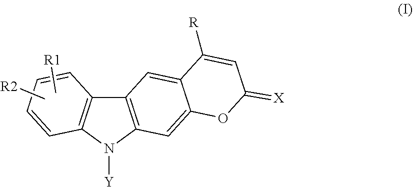

in which formula I:

R is a C1-C3 alkyl group, a C1-C3 alkyl halide group, a substituted or unsubstituted phenyl group, or a substituted or unsubstituted cyclohexyl group;

X is an oxygen atom or a sulfur atom;

R1 and R2 are each independently a group selected from the group consisting of a hydrogen atom, a halogen atom, a —OH group, an amino group, a nitro group, a methyl group, a methyl fluoride group, an ethyl group, an ethyl fluoride group, and a C1-C3 alkylsulfanyl group; and

Y represents a hydrogen atom; a saccharide including ribose and deoxyribose; a polysaccharide including a polyribose chain and a polydeoxyribose chain of a nucleic acid; a polyether; a polyol; an alkanolamine; an amino acid; a polypeptide chain including a polypeptide chain of a peptide nucleic acid; or a water-soluble synthetic polymer,

wherein the method comprises the step of:

causing a compound of the following formula II:

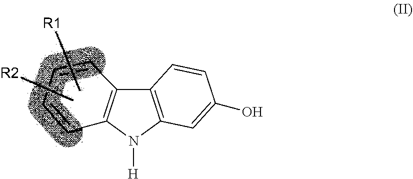

in which formula II, R1 and R2 are independently groups defined as R1 and R2 in the formula I, respectively,
and a compound of the following formula III:

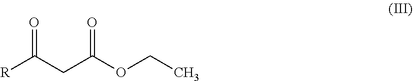

in which formula III, R is a group defined as R in the formula I,
to undergo a Pechmann condensation reaction in a presence of an organic solvent and an acid catalyst to provide a compound of the following formula IV:

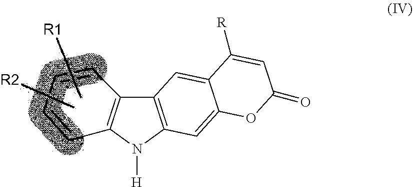

in which formula IV,

R is a group defined as R in the formula I; and

R1 and R2 are independently groups defined as R1 and R2 in the formula I, respectively.

(2)

The method according to (1), wherein the group Y in the formula I is a group selected from the group consisting of atoms and groups represented by the following (i) to (iv):

(i) a hydrogen atom;

(ii) a group represented by the following formula Ya:

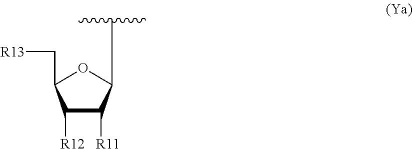

in which formula Ya:

R11 is a hydrogen atom or a hydroxyl group,

R12 is a hydroxyl group or a —O-Q₁ group,

R13 is a hydroxyl group or a —O-Q₂ group,

Q₁ is a group selected from the group consisting of:
a phosphate group formed together with 0 bonded to Q₁;
a nucleotide, nucleic acid or peptide nucleic acid linked via a phosphodiester bond formed by a phosphate group formed together with O bonded to Q₁; and
a protecting group selected from:
a trityl group, a monomethoxytrityl group, a dimethoxytrityl group, a trimethoxytrityl group, a trimethylsilyl group, a triethylsilyl group, a t-butyldimethylsilyl group, an acetyl group, and a benzoyl group;

Q₂ is a group selected from the group consisting of:
a phosphate group formed together with 0 bonded to Q₂;
a nucleotide, nucleic acid or peptide nucleic acid linked via a phosphodiester bond formed by a phosphate group formed together with O bonded to Q₂; and
a protecting group selected from:
a 2-cyanoethyl-N,N-dialkyl(C1-C4)phosphoramidite group, a methylphosphonamidite group, an ethylphosphonamidite group, an oxazaphospholidine group, a thiophosphite group, a TEA salt of —PH(═O)OH, a DBU salt of —PH(═O)OH, a TEA salt of —PH(═S)OH, and a DBU salt of —PH(═S)OH;
(iii) a group represented by the following formula Yb:

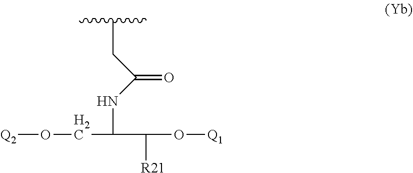

in which formula Yb:

R21 represents a hydrogen atom, a methyl group, or an ethyl group;

Q1 is a group defined as Q1 in the formula Ya; and

Q2 is a group defined as Q2 in the formula Ya; and

(iv) a group represented by the following formula Yc:

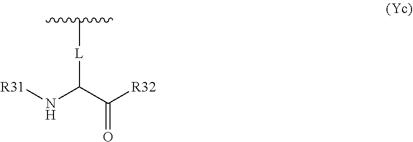

in which formula Yc:

R31 represents a protecting group for the amino group, a hydrogen atom, or a polypeptide linked via a peptide bond formed together with NH bonded to R31;

R32 represents a hydroxyl group, or a polypeptide linked via a peptide bond formed together with CO bonded to R32; and

L is a linker moiety or a single bond.

(3)

A compound of the following formula I:

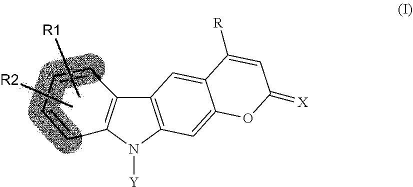

in which formula I:

R is a C1-C3 alkyl group, a C1-C3 alkyl halide group, a substituted or unsubstituted phenyl group, or a substituted or unsubstituted cyclohexyl group;

X is an oxygen atom or a sulfur atom;

R1 and R2 are each independently a group selected from the group consisting of a hydrogen atom, a halogen atom, a —OH group, an amino group, a nitro group, a methyl group, a methyl fluoride group, an ethyl group, an ethyl fluoride group, and a C1-C3 alkylsulfanyl group; and

Y represents a hydrogen atom; a saccharide including ribose and deoxyribose; a polysaccharide including a polyribose chain and a polydeoxyribose chain of a nucleic acid; a polyether; a polyol; an alkanolamine; an amino acid; a polypeptide chain including a polypeptide chain of a peptide nucleic acid; or a water-soluble synthetic polymer.

(4)

The compound according to (3), wherein the group Y in the formula I is a group selected from the group consisting of atoms and groups represented by the following (i) to (iv):

(i) a hydrogen atom;

(ii) a group represented by the following formula Ya:

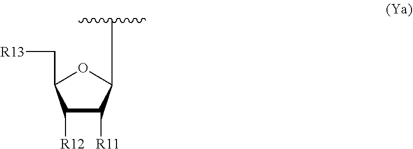

in which formula Ya:

R11 is a hydrogen atom or a hydroxyl group,

R12 is a hydroxyl group or a —O-Q₁ group,

R13 is a hydroxyl group or a —O-Q₂ group,

Q1 is a group selected from the group consisting of:
a phosphate group formed together with 0 bonded to Q1;
a nucleotide, nucleic acid or peptide nucleic acid linked via a phosphodiester bond formed by a phosphate group formed together with O bonded to Q₁; and
a protecting group selected from:
a trityl group, a monomethoxytrityl group, a dimethoxytrityl group, a trimethoxytrityl group, a trimethylsilyl group, a triethylsilyl group, a t-butyldimethylsilyl group, an acetyl group, and a benzoyl group;

Q2 is a group selected from the group consisting of:
a phosphate group formed together with O bonded to Q2;
a nucleotide, nucleic acid or peptide nucleic acid linked via a phosphodiester bond formed by a phosphate group formed together with O bonded to Q2; and
a protecting group selected from:
a 2-cyanoethyl-N,N-dialkyl(C1-C4)phosphoramidite group, a methylphosphonamidite group, an ethylphosphonamidite group, an oxazaphospholidine group, a thiophosphite group, a TEA salt of —PH(═O)OH, a DBU salt of —PH(═O)OH, a TEA salt of —PH(═S)OH, and a DBU salt of —PH(═S)OH;

(iii) a group represented by the following formula Yb:

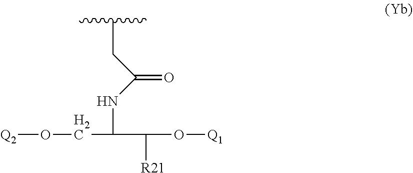

in which formula Yb:

R21 represents a hydrogen atom, a methyl group, or an ethyl group;

Q1 is a group defined as Q1 in the formula Ya; and

Q2 is a group defined as Q2 in the formula Ya; and

(iv) a group represented by the following formula Yc:

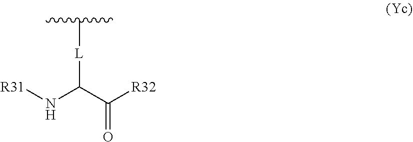

in which formula Yc:

R31 represents a protecting group for the amino group, a hydrogen atom, or a polypeptide linked by a peptide bond formed together with NH bonded to R31;

R32 represents a hydroxyl group, or a polypeptide linked by a peptide bond formed together with CO bonded to R32; and

L is a linker moiety or a single bond.

(5)

A photoreactive crosslinking agent comprising the compound according to (3) or (4).

(6)

A method for forming a photocrosslink between nucleic acid bases each having a pyrimidine ring, using the compound according to (3) or (4).

(7)

A method comprising:

producing the compound of the formula I by the step of causing the compound of the formula II and the compound of the formula III to undergo the Pechmann condensation reaction in the presence of the organic solvent and the acid catalyst to provide the compound of the above formula IV; and

forming a photocrosslink between nucleic acid bases each having a pyrimidine ring, using the compound of the formula I.

### Advantageous Effects of Invention

According to the present invention, a novel photoreactive compound that can be used in a photoreaction technique for a nucleic acid can be synthesized in a short period of time with good yield.

### BRIEF DESCRIPTION OF THE DRAWINGS

FIG. 1 is a synthesis scheme (scheme 1) for a nucleoside analog (^{MEP}K);

FIG. 2 is an MS spectrum of an oligonucleic acid containing ^{MEP}K;

FIG. 3A is a chromatogram of a crosslinked sample at light irradiation 0 sec;

FIG. 3B is a chromatogram of a crosslinked sample at light irradiation 60 sec;

FIG. 3C is a view for explaining a photocrosslinking reaction;

FIG. 4 is an MS spectrum of a photocrosslinked product;

FIG. 5 is a synthesis scheme (scheme 2) for a nucleoside analog (^{MEP}D);

FIG. 6 is a synthesis scheme (scheme 3) for a nucleoside analog (^{MEP}A);

FIG. 7 is a synthesis scheme (scheme 4) for a nucleoside analog (^{PC}X);

FIG. 8A is a schematic explanatory view of a procedure for synthesizing a nucleoside analog (^{PC}X);

FIG. 8B is a structure of an isomer that will be a by-product;

FIG. 9 is a schematic explanatory view of a procedure for synthesizing a nucleoside analog (^{PC}X); and

FIG. 10 is an explanatory view for comparing the synthesis of ^{MEP}K, ^{MEP}D, and ^{MEP}A.

### DETAILED DESCRIPTION OF THE INVENTION

The present invention will be described below in detail by providing specific embodiments. The present invention is not limited to the following specific embodiments as mentioned below.

### [Method of Producing Photoreactive Compound]

Production of a photoreactive compound according to the present invention is carried out by a method for producing a compound of the following formula I:

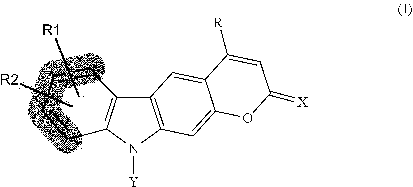

the method comprising the step of:

causing a compound of the following formula II:

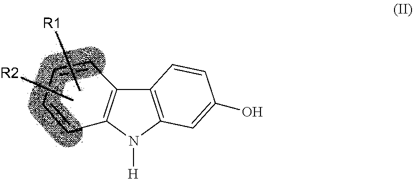

and a compound of the following formula III:

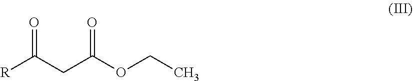

to undergo a Pechmann condensation reaction in a presence of an organic solvent and an acid catalyst to provide:
a compound of the following formula IV:

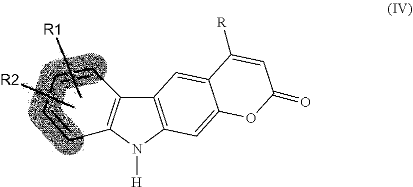

### [R in Formula I]

In a preferred embodiment, R in the formula I may be a C1-C3 alkyl group, a C1-C3 alkyl halide group, a substituted or unsubstituted phenyl group, or a substituted or unsubstituted cyclohexyl group.

In a preferred embodiment, the alkyl group can be, for example, a C1-C3 alkyl group, preferably a C1-C2 alkyl group, including, for example, a methyl group and an ethyl group. In a preferred embodiment, the alkyl halide group can be, for example, a C1-C3 alkyl halide group, preferably a C1-C2 alkyl halide group. Examples of the halogen include Br, Cl, F and I. For halogenation, the hydrogen atom of the alkyl group is substituted with the halogen atom, and the number of substitutions can be one or more, for example, one, two, or three. In a preferred embodiment, the phenyl group may be substituted or unsubstituted, and for example, the hydrogen atom of the phenyl group can be substituted with a C1-C2 alkyl or halogen atom, and the number of substitutions may be one or more, for example one, two, or three. In a preferred embodiment, the cyclohexyl group may be substituted or unsubstituted, and for example, the hydrogen atom of the cyclohexyl group can be substituted with a C1-C2 alkyl or halogen atom, and the number of substitutions may be one or more, for example one, two, or three.

### [X in Formula I]

In a preferred embodiment, X in the formula I can be an oxygen atom or a sulfur atom, preferably an oxygen atom.

### [R1 and R2 in Formula I]

R1 and R2 in the formula I can be each independently a group selected from the group consisting of a hydrogen atom, a halogen atom, a —OH group, an amino group, a nitro group, a methyl group, a methyl fluoride group, an ethyl group, an ethyl fluoride group, and a C1-C3 alkylsulfanyl group.

In a preferred embodiment, examples of the halogen atom include Br, Cl, F, and I atoms. Examples of the methyl fluoride group include —CH₂F, —CHF₂, and —CF₃. Examples of the ethyl fluoride group include —CH₂—CH₂F, —CH₂—CHF₂, —CH₂—CF₃, —CHF—CH₃, —CHF—CH₂F, —CHF—CHF₂, —CHF—CF₃, —CF₂—CH₃, —CF₂—CH₂F, —CF₂—CHF₂, and —CF₂—CF₃. Examples of the C1-C3 alkylsulfanyl group include —CH₂—SH, —CH₂—CH₂—SH, —CH(SH)—CH₃, —CH₂—CH₂—CH₂—SH, —CH₂—CH(SH)—CH₃ and —CH(SH)—CH₂—CH₃ groups. In a preferred embodiment, R1 and R2 can each independently be a hydrogen atom, a halogen atom, a —NH₂ group, a —OH group, a —CH₃ group, and preferably a hydrogen atom.

In a preferred embodiment, R2 can be a hydrogen atom while at the same time R1 can be the group as defined above.

In a preferred embodiment, when in the 6-membered ring at the left end in the formula I, the carbon atom to which the nitrogen atom is linked is numbered as a C1 position, and the carbon atoms of the 6-membered ring are sequentially numbered as C2, C3, C4, C5, and C6 positions clockwise, R1 and R2 can each independently be a substituent for the carbon atom at any position of C2, C3, C4, and C5 positions. In a preferred embodiment, R1 and R2 can be substituents at the C3 and C4 positions, respectively. In a preferred embodiment, R1 can be a substituent at the C3 position and R2 can be a hydrogen atom at the C4 position.

### [Compound of Formula I′]

In a preferred embodiment, the compound of the formula I can be a compound represented by the following formula I′:

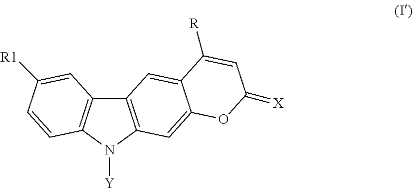

In the formula I′, R, R1, X, and Y represent the groups defined in the formula I.

### [Y in Formula I]

Y can be a hydrogen atom; a saccharide including ribose and deoxyribose; a polysaccharide including a polyribose chain and a polydeoxyribose chain of a nucleic acid; a polyether; a polyol; a polypeptide chain including a polypeptide chain of a peptide nucleic acid; or a water-soluble synthetic polymer.

In a preferred embodiment, Y can be a hydrogen atom, and in this case, the compound of the formula I is a compound represented by the following formula IV.

### [Group Represented by Formula Ya]

In a preferred embodiment, Y can be a group represented by the following formula Ya, in which case the compound of formula I will be a compound represented by the following formula V.

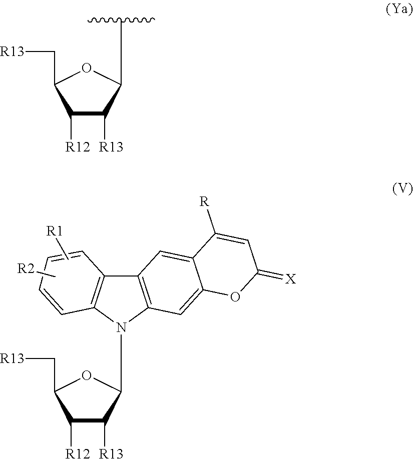

### [Substituents of Formula V]

In the formula V, R, R1, R2, and X represent the groups defined in the formula I, and R11, R12, and R13 represent the groups defined in the formula Ya.

### [R11, R12, R13 of Formula Ya]

In the formula Ya, R11 is a hydrogen atom or a hydroxyl group, R12 is a hydroxyl group or a —OQ₁ group, and R13 is a hydroxyl group or a —OQ₂ group.

The above Q₁ can be a group selected from the group consisting of: a phosphate group formed together with O bonded to Q₁;

a nucleotide, nucleic acid or peptide nucleic acid linked via a phosphodiester bond formed by a phosphate group formed together with O bonded to Q₁; and
a protecting group selected from:
a trityl group, a monomethoxytrityl group, a dimethoxytrityl group, a trimethoxytrityl group, a trimethylsilyl group, a triethylsilyl group, a t-butyldimethylsilyl group, an acetyl group, and benzoyl group.

The above Q₂ can be a group selected from the group consisting of: a phosphate group formed together with O bonded to Q2;

a nucleotide, nucleic acid or peptide nucleic acid linked via a phosphodiester bond formed by a phosphate group formed together with O bonded to Q2; and a protecting group selected from:
a 2-cyanoethyl-N,N-dialkyl(C1-C4)phosphoramidite group, a methylphosphonamidite group, an ethylphosphonamidite group, an oxazaphospholidine group, a thiophosphite group, a TEA salt of —PH(═O)OH, a DBU salt of —PH(═O)OH, a TEA salt of —PH(═S)OH, and a DBU salt of —PH(═S)OH.

The 2-cyanoethyl-N,N-dialkyl(C1-C4)phosphoramidite group has the following structure:

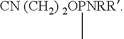

Each of the groups R and R′ forming the dialkyl group as described above can be a C1-C4 alkyl group. Examples of such a 2-cyanoethyl-N,N-dialkyl(C1-C4)phosphoramidite group include a 2-cyanoethyl-N,N-dimethylphosphoramidite group, a 2-cyanoethyl-N,N-diethylphosphoroamidite group and a 2-cyanoethyl-N,N-diisopropylphosphoramidite group.

The methylphosphonamidite group has the following structure:

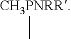

Each of the groups R and R′ as described above can be a hydrogen atom or a C1-C4 alkyl group.

The ethylphosphonamidite group has the following structure:

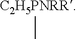

Each of the groups R and R′ can be a hydrogen atom or a C1-C4 alkyl group.

The oxazaphospholidine group has the following structure:

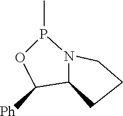

and also includes a substituted body in which the hydrogen atom is substituted by a C1 to C4 alkyl group, in the above structure.

The thiophosphite group has the following structure:

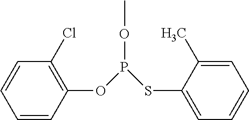 [SMILES: COP(Oc1ccccc1Cl)Sc1ccccc1C]

and also includes a substituted body in which the hydrogen atom is substituted by a C1-C4 alkyl group, in the above structure.

Each of the TEA salt of —PH(═O)OH and the TEA salt of —PH(═S)OH is a triethylamine (TEA) salt of each.

Each of the DBU salt of —PH(═O)OH and the DBU salt of —PH(═S)OH is a diazabicycloundecene (DBU) salt of each.

In a preferred embodiment, Q₁ can be a nucleotide or nucleic acid linked via a phosphodiester bond formed by a phosphate group formed together with O bonded to Q₁.

In a preferred embodiment, Q₁ can be the protecting group as described above, preferably a dimethoxytrityl group, a trityl group, a monomethoxytrityl group, a trimethoxytrityl group, and particularly preferably the dimethoxytrityl group.

In a preferred embodiment, Q2 can be a nucleotide or nucleic acid linked via a phosphodiester bond formed by a phosphate group formed together with O bonded to Q2.

In a preferred embodiment, Q2 can be the protecting group as described above, preferably a 2-cyanoethyl-N,N-dialkyl(C1-C4)phosphoramidite group, an oxazaphospholidine group, and a thiophosphite group, and more particularly preferably 2-cyanoethyl-N,N-diisopropylphosphoramidite group.

In a preferred embodiment, in the formula Ya, R11 can be a hydrogen atom, R12 can be a hydroxyl group, and R13 can be a hydroxyl group. That is, Y can be deoxyribose.

In a preferred embodiment, in the formula Ya, R11 can be a hydroxyl group, R12 can be a hydroxyl group, and R13 can be a hydroxyl group. That is, Y can be ribose.

### [Group Represented by Formula Yb]

In a preferred embodiment, Y can be a group represented by the following formula Yb, in which case the compound of formula I will be a compound represented by the following formula VI.

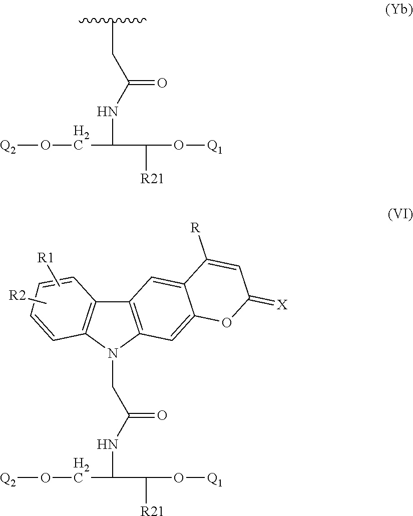

### [Substituents of Formula VI]

In the formula VI, R, R1, R2, and X represent the groups defined in the formula I, and R21, Q1, and Q2 represent the groups defined in the formula Yb.

### [R21, Q1, and Q2 of Formula Yb]

In the formula Yb, R21 represents a hydrogen atom, a methyl group, or an ethyl group, Q1 can be a group defined as Q1 in the formula Ya, and Q2 can be a group defined as Q2 in the formula Ya.

In a preferred embodiment, the skeleton structure represented by the following formula Yb1:

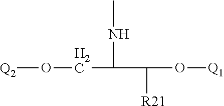

which is included in the formula Yb, can be D-threoninol structure represented by the following formula:

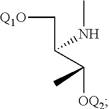

L-threoninol structure represented by the following formula:

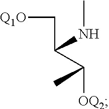

or,
a serinol structure represented by the following formula:

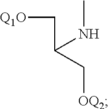

### [Group Represented by Formula Yc]

In a preferred embodiment, Y can be a group represented by the following formula Yc, in which case the compound of formula I will be a compound represented by the following formula VII.

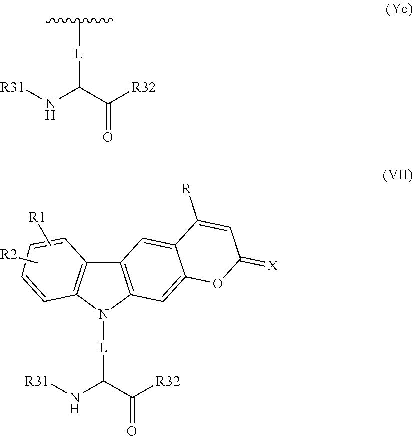

### [Substituents of Formula VII]

In the formula VII, R, R1, R2, and X represent the groups defined in formula I, and R31, R32, and L represent the groups defined in the formula Yc.

### [R31, R32, L of Formula Yc]

In the formula Yc, R31 represents a protecting group for the amino group, a hydrogen atom, or a polypeptide linked via a peptide bond formed together with NH bonded to R31,

R32 represents a hydroxyl group, or a polypeptide linked via a peptide bond formed together with CO bonded to R32, and

L is a linker moiety or a single bond.

In a preferred embodiment, an alkanediyl group can be used as the linker moiety which is L. Examples of the alkanediyl group include a C1-C3 alkanediyl group, preferably a C1-C2 alkanediyl group, and particularly preferably a methylene group and an ethylene group.

In a preferred embodiment, L can be a methylene group, an ethylene group or a single bond. The case where L is the single bond means a state where N and C, which are bonded to L, are bonded by a single bond.

The protecting group for the amino group can include a protecting group known as the protecting group for the amino group. In a preferred embodiment, the protecting group for the amino group that can be used includes a protecting group selected from the group consisting of a fluorenylmethoxycarbonyl group (Fmoc), a tert-butoxycarbonyl group (Boc), a benzyloxycarbonyl group (Cbz), and an allyloxycarbonyl group (Alloc).

In a preferred embodiment, in the formula Yc, R31 can be a hydrogen atom, R32 can be a hydroxyl group, and L can be a single bond. That is, Y can be an amino acid.

### [Photoreactive Nucleoside Analog]

The compound represented by the above formula I is a photoreactive nucleotide analog (photocrosslinkable modified nucleoside) in which a base moiety is substituted with a photoreactive artificial base, When Y is Ya and the compound is ribose or deoxyribose. This can be incorporated into nucleic acids by known means used in natural nucleosides to prepare photocrosslinkable modified nucleic acids.

In the compound represented by the above formula I in which Y is Yb, the moiety of the sugar skeleton of a ribose (or a deoxyribose) structure in the case of a natural nucleoside is replaced with the skeleton structure represented by the above formula Yb. Therefore, this compound can also be referred to as a photoreactive nucleotide analog (photocrosslinkable modified nucleoside) in which the base moiety is substituted with the photoreactive artificial base. Surprisingly, despite these differences in skeletal structures, the photoreactive nucleotide analog can be handled as in the photoreactive nucleotide analog in which Y is Ya and it is ribose or deoxyribose, in terms of incorporation into nucleic acids and photoresponsivity. The photoreactive nucleotide analog can be incorporated into a nucleic acid by a known means used in a natural nucleoside to prepare a photocrosslinkable modified nucleic acid.

### [Photoreactive Artificial Amino Acid]

The compound represented by the above formula I in which Y is Yc and it is an amino acid can be referred to as a photoreactive artificial amino acid having a photoreactive artificial base structure. Since the photoreactive artificial amino acid is the amino acid, it is incorporated into a polypeptide chain by a known means used in natural amino acids to prepare a photoreactive artificial polypeptide (photocrosslinkable modified polypeptide).

### [Compound of Formula II]

In the formula II, R1 and R2 can be R1 and R2 defined in the formula I, respectively.

### [Compound of Formula II′]

In a preferred embodiment, the compound of the formula II can be a compound represented by the following formula II′:

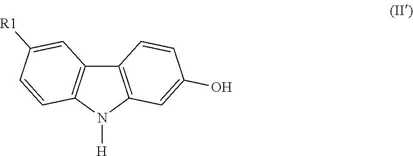

### [Compound of Formula III]

In the formula III, R can be the R defined in the formula I. However, in terms of the progress of the Pechmann condensation reaction, R must not be a hydrogen atom.

### [Pechmann Condensation Reaction]

In the production method of the present invention, the compound of the formula III is condensed to the compound of the formula II by the Pechmann condensation reaction to synthesis the compound of the formula IV. The Pechmann condensation reaction forms a ring so as to change the tricyclic structure to the tetracyclic structure. In a preferred embodiment, the Pechmann condensation reaction is carried out by heating in a presence of an organic solvent and an acid catalyst. As the organic solvent, preferably a C1-C3 alcohol, and more preferably ethanol, can be used. As the acid catalyst, a sulfuric acid catalyst is preferably used. The heating temperature is, for example, 70° C. or more, and preferably 80° C. or more, and more preferably 85° C. or more.

In the production method according to the present invention, the compound of formula IV can be synthesized in an extremely short period of time with an extremely high yield, by condensing the compound of the formula III to the compound of the formula II by the Pechmann condensation reaction. Therefore, the compound of the formula I can be dramatically and efficiently synthesized.

### [Compound of Formula IV]

In the formula IV, R, R1 and R2 can be R, R1 and R2 defined in the formula I, respectively.

In the compound of the formula IV, Y in the formula I is a hydrogen atom. The hydrogen atom at the Y position can be substituted by a known means to form the group defined as Y in the formula I. That is, after the step of causing the Pechmann condensation reaction, a step of substituting H on the NH group of the compound in the formula IV with Y to prepare the compound of the formula I can be carried out. However, when Y in the formula I is a hydrogen atom, such a substitution step is not necessary as a matter of course.

In the present invention, the present inventors believe that the reason why the compound of the formula I can be dramatically and efficiently synthesized would be that the compound of the formula IV is synthesized with an extremely high yield to produce a decreased amount of by-products, and as a result, the subsequent side reactions are extremely reduced, resulting in the efficient overall reaction leading to the compound of the formula I.

### [Compound of Formula IV]

In a preferred embodiment, the compound of the formula IV can be a compound represented by the following formula IV′. When the compound of the formula II′ is used in the Pechmann condensation, the compound represented by the formula IV′ is obtained:

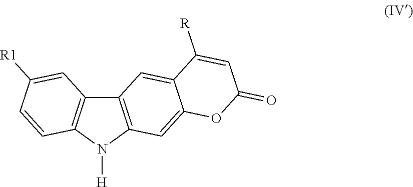

### [Photoreactive Modified Nucleic Acid]

In a preferred embodiment, the compound of the formula I can be a photoreactive nucleoside analog, which is introduced into the nucleic acid via a phosphodiester bond to provide a photoreactive modified nucleic acid.

### [Reagent for Producing Modified Nucleic Acid]

In a preferred embodiment, the compound of the formula I can be used to produce a photoreactive modified nucleic acid by introducing it into the nucleic acid via a phosphodiester bond. That is, the compound of the formula I can be used as a reagent for producing a modified nucleic acid. In order to have a reagent for producing a modified nucleic acid, the reagent may be in the form of a reagent that can be used by a known nucleic acid synthesis means. For example, it is possible to have a reagent for synthesizing a modified nucleic acid (a monomer for synthesizing a modified nucleic acid) that can be used by, for example, a phosphoramidite method and a H-phosphonate method.

### [Photoreactive Crosslinker]

In a preferred embodiment, the pyranocarbazole moiety of the compound of the formula I can form a crosslink by photoreaction. When the compound of the formula I is formed as a single-stranded modified nucleic acid, it can form a double helix with a complementary single-stranded nucleic acid, and the pyranocarbazole moiety can form a crosslink by photoreaction, so that a photocrosslink between the strands is formed from one strand of the double helix to the other strand. That is, the compound of the formula I can be used as a photoreactive crosslinker.

### [Formation of Photocrosslink]

In a preferred embodiment, when the photoreactive modified nucleic acid is used as a single-stranded nucleic acid, it can hybridize with a complementary single-stranded nucleic acid to form a double helix. In the formation of the double helix, the nucleic acid bases at positions where base pairs should be formed in the complementary strand with methylpyranocarbazole structure portion can be freely selected without any particular limitation. When the formed double helix is irradiated with light, a crosslink can be formed by a photoreaction between the nucleic acid strands forming the double helix. The photocrosslink is formed between a nucleic acid base and the methylpyranocarbazole structure, the nucleic acid base being located at a position where a base pair is formed in the complementary strand, with a nucleic acid base located on the 5′ terminal side by one base in the sequence from a position where the methylpyranocarbazole structural moiety is located as a nucleic acid base. In other words, the photocrosslink is formed between a nucleic acid base and the methylpyranocarbazole structure, the nucleic acid base being located at the 3′ terminal side by one base in the sequence from a nucleic acid base at a position where a base pair should be formed with the methylpyranocarbazole structural moiety in the complementary strand.

### [Base Specificity of Photocrosslinking]

In a preferred embodiment, the counterpart base with which the methylpyranocarbazole structure can form a photocrosslink is a base having a pyrimidine ring. On the other hand, the methylpyranocarbazole structure does not form a photocrosslink with a base having a purine ring. In other words, the photocrosslinkable compound according to the present invention has specificity that it forms photocrosslinks with cytosine, uracil, and thymine as natural nucleic acid bases, whereas it does not form photocrosslinks with guanine and adenine.

### [Sequence Selectivity of Photoreactive Crosslinker]

In a preferred embodiment, the photoreactive modified nucleic acid (photocrosslinkable modified nucleic acid) can be photocrosslinked after hybridizing with a sequence having a base sequence complementary to the modified nucleic acid to form a double helix. This can allow a photocrosslinking reaction to be performed only on the target specific sequence. In other words, the photoreactive crosslinker according to the present invention can provide very high base sequence selectivity by designing a sequence as needed.

### [Wavelength of Light Irradiation]

A wavelength of light irradiated for photocrosslinking can be, for example, in a range of from 350 to 600 nm, and preferably in a range of from 400 to 600 nm, and more preferably in a range of from 400 to 550 nm, and even more preferably in a range of from 400 to 500 nm, and still more preferably in a range of from 400 to 450 nm. In particular, light containing a wavelength of 400 nm is preferable. In a preferred embodiment, single wavelength laser light in these wavelength ranges can be used. Thus, in the present invention, a photocrosslink can be formed by irradiation with light having a wavelength in the visible light region. The conventional photoreactive crosslinkers require irradiation with light having a wavelength shorter than these ranges. According to the present invention, a photocrosslink can be formed by irradiation with light having a longer wavelength than the conventional photoreactive crosslinkers, which is advantageous in that adverse effects on nucleic acids and cells due to light irradiation can be minimized.

### [Photoreaction Time]

The photocrosslinking according to the present invention proceeds very rapidly. For example, in a case of psoralen known as a photoreactive compound, the photoreaction requires several hours (by irradiation with light having 350 nm), whereas, in the present invention, the photoreaction proceeds by irradiation with light having a much longer wavelength, for example, for only 10 seconds to 60 seconds (by irradiation with light having 400 nm) to causes photocrosslinking. That is, by using the photocrosslinker according to the present invention, the photoreaction can be allowed to proceed by irradiation with light, for example, for 1 to 120 seconds, or for 1 to 60 seconds, to form a photocrosslink.

### [Photoreaction Temperature]

In a preferred embodiment, to proceed with the photocrosslinking reaction, irradiation with light is generally carried out at a temperature in a range from 0 to 50° C., and preferably from 0 to 40° C., and more preferably from 0 to 30° C., and even more preferably from 0 to 20° C., and still more preferably from 0 to 10° C., and still more preferably from 0 to 5° C.

### [Photoreaction Conditions]

In a preferred embodiment, due to the use of photoreaction, the photocrosslinking has no particular restriction on a pH, a salt concentration or the like, and can be carried out by irradiation with light in a solution having a pH and a salt concentration where biopolymers such as nucleic acids can be stably present.

### [Photoreactive Artificial Polypeptide]

In a preferred embodiment, the compound of the formula I can be a photoreactive artificial amino acid, which is introduced into an amino acid sequence of a polypeptide chain via a peptide bond to provide a photoreactive artificial polypeptide (photocrosslinkable modified polypeptide). Since the photoresponsiveness of the photoreactive artificial amino acid is maintained even if it is introduced into the polypeptide chain, the resulting polypeptide is the photoreactive artificial polypeptide, even if the photoreactive artificial amino acid has been introduced into any polypeptide chain having any amino acid sequence.

### [Reagent for Producing Photoreactive Artificial Polypeptide]

The photoreactive artificial amino acid(s) can be introduced into a polypeptide chain by a known means. That is, in a known polypeptide chain synthesis means, peptide synthesis may be carried out using the photoreactive artificial amino acid(s) in place of the natural amino acid(s) or the like. The photoreactive artificial amino acid can optionally be protected by known protecting groups and subjected to peptide synthesis. Examples of such a peptide synthesis means include a Fmoc peptide solid phase synthesis method and a Boc peptide solid phase synthesis method. Therefore, the compound of formula I can be used as a reagent for producing the photoreactive artificial polypeptide in a desired form thereof.

### Examples

Hereinafter, the present invention will be described in detail with reference to Examples. The present invention is not limited to Examples illustrated below.

### [Synthesis of Nucleoside Analog (^{MEP}K)]

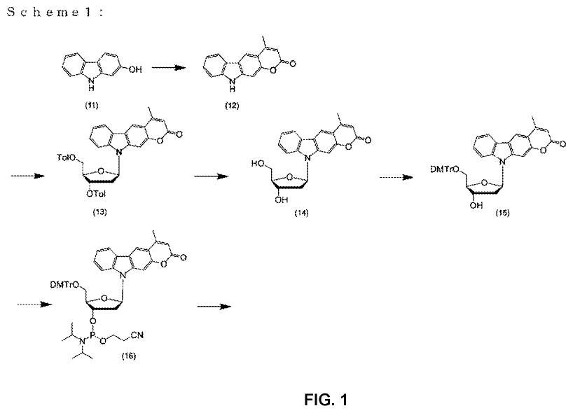

A photoresponsive artificial nucleoside analog molecule (which may be referred to as a nucleoside analog, or a photoreactive element, or a photocrosslinking element) (^{MEP}K) was synthesized along the synthetic route as shown in Scheme 1 of FIG. 1, and a modified nucleic acid synthetic monomer was further synthesized to form a modified DNA into which the monomer was introduced. Details for each step will be described later.

In each synthesis step of Scheme 1, the conditions (a) to (e) are as follows. The symbol “r.t.” means room temperature.

(a) Ethyl acetoacetate, H₂SO₄, EtOH, 90° C., 2 h;

### (b) KOH, TDA-1, Chlorosugar, CH₃CN, r.t., 8 h;

(c) NaOCH₃, CH₃OH, CHCl₃, r.t., 10 h;

### (d) DMTrCl, DMAP, Pyridine, r.t., 24 h; and

(e) (iPr₂N)₂PO(CH₂)₂CN, tetrazole, CH₃CN, r.t., 4 h.

### [Synthesis of Compound 12]

Compound 11 (5.00 g, 27.3 mmol), ethyl acetoacetoate (3.79 mL, 30.0 mmol) and EtOH (30 mL) were placed in an eggplant flask and stirred on ice. To the mixture, conc. H₂SO₄ (7 mL) was dropped. EtOH (10 mL) was added thereto, and stirred at 90° C. for 2 hours. Disappearance of the raw materials was confirmed by TLC (CHCl₃:MeOH=9:1), and the stirring was stopped. Acetone was added to the solution to perform recrystallization. The compound obtained by recrystallization was filtered, washed with chloroform, and then dried to obtain Compound 12 (4.90 g, 19.6 mmol, 72%).

¹H-NMR (400 MHz, DMSO-d₆) δ 11.64 (s, 1H), 8.53 (s, 1H), 8.25 (d, 1H, J=7.68 Hz), 7.53 (d, 1H, 8.00 Hz), 7.44 (t, 1H, J=7.56 Hz), 7.40 (s, 1H), 7.24 (t, 1H, J=7.36 Hz), 6.26 (s, 1H), 2.59 (s, 3H) SALDI-MS: Calc'd for C₁₆H₁₁NNaO₂ [M+Na]⁺=272.0681, Found 272.0682.

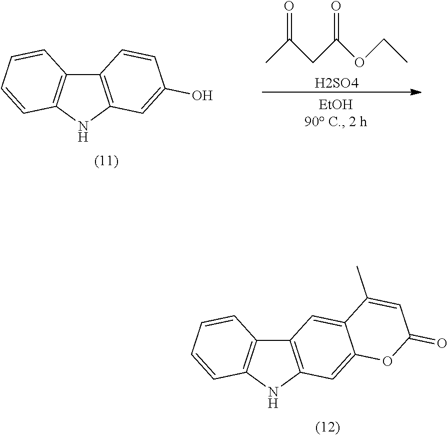

### [Synthesis of Compound 13]

Compound 12 (300 mg, 1.20 mmol) and KOH (260 mg, 10.1 mmol) were added and purged with N₂. CH₃CN (50 mL) and TDA-1 (250 μL) were added and stirred. After 30 minutes, chlorosugar (1.17 g, 3.00 mmol) was added and stirred at room temperature for 6 hours. It was confirmed by TLC (CHCl₃) and the reaction was stopped. The resulting precipitate was removed by suction filtration, and the solvent was removed from the filtrate by an evaporator.

¹H-NMR (400 MHz, DMSO-d₆) δ 11.67 (s, 1H), 8.47 (s, 1H), 8.17 (dt, 2H, J=11.4 Hz), 7.50-7.43 (m, 2H), 7.25 (dt, 1H, J=8.54 Hz), 6.33 (d, 1H, J=4.75 Hz), SALDI-MS: Calc'd for C₁₆H₁₁NNaO₂ [M+Na]⁺=272.0681, Found 272.0682.

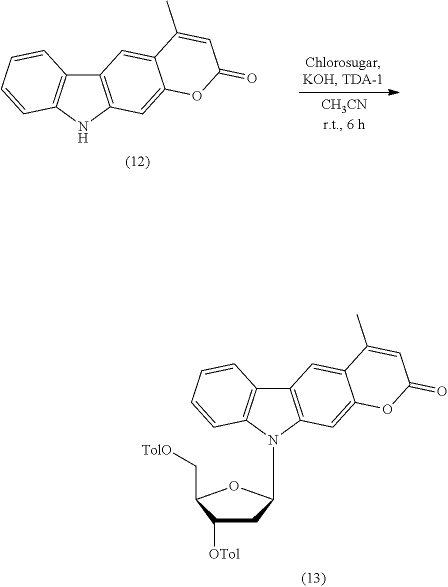

### [Synthesis of Compound 14]

Methanol (40 mL) and CHCl₃ (30 mL) were added to an eggplant flask containing Compound 3 (1.58 g, 3.82 mmol), and NaOMe (1.00 g) was added and stirred at room temperature for 10 hours. The solvent was then removed by an evaporator, and the residue was purified by column chromatography (CHCl₃:MeOH=9:1). After purification, the resulting product was dried to obtain Compound 4 (360 mg, 0.985 mmol, 82%).

¹H-NMR (400 MHz, DMSO-d₆) δ 8.62 (d, 1H, 3.04 Hz), 8.30 (d, 1H, 7.68 Hz), 7.89-7.80 (m, 2H), 7.48 (t, 1H, 7.78 Hz), 7.31 (t, 1H, J=5.96 Hz), 6.71 (t, 1H, J=7.8 Hz), 6.30 (s, 1H), 5.42 (s, 1H), 5.16 (s, 1H), 4.50 (d, 1H, 3.44 Hz), 3.89 (d, 1H, 3.72 Hz), 3.78 (s, 2H), 2.59 (s, 3H), 2.17-2.12 (m, 1H), 1.14-1.06 (m, 1H) SALDI-MS: Calc'd for C₂₁H₁₉NNaO₅ [M+Na]⁺=388.1155, Found 388.1152.

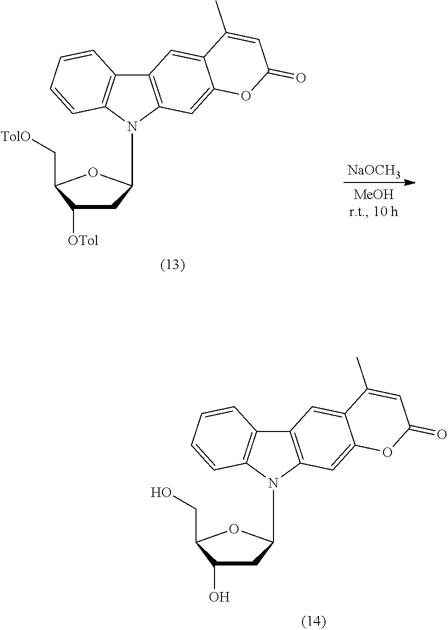

### [Synthesis of Compound 15]

Compound 4 (375 mg, 1.03 mmol) and DMAP (12.3 mg, 0.101 mmol) were added to an eggplant flask, purged with N₂. Dry pyridine (10 ml) was then added on an ice bath. DMTrCl (525 mg, 1.55 mmol) was added. The resulting mixture was then stirred at room temperature for 24 hours. After confirming the disappearance of the raw materials by TLC (CHCl₃:MeOH=9:1), the reaction solution was concentrated by an evaporator. The concentrated solution was subjected to azeotrope several times with Toluene. This was then purified by column chromatography (CHCl₃:MeOH=19:1) to obtain white powder (128 mg, 0.192 mmol, 18.6%).

¹H-NMR (400 MHz, DMSO-d₆) δ SALDI-MS: Calc'd for C₂₁H₁₉NNaO₅ [M+Na]⁺=, Found.

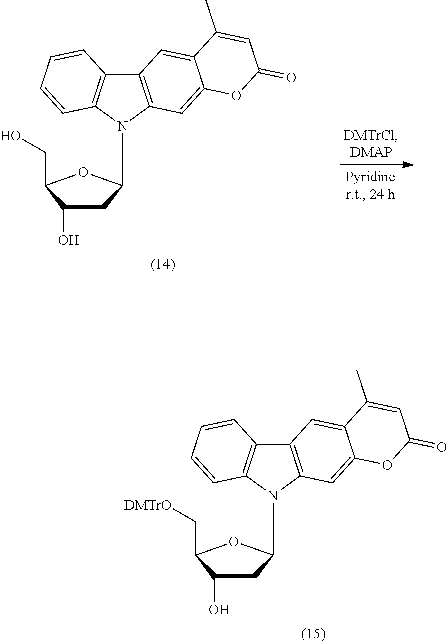

### [Synthesis of Compound 16]

CH₂Cl₂ (4.17 mL) was added to Compound 15 (129 mg, 0.192 mmol) in an eggplant flask in N₂. Then, 0.25M of tetrazole (800 μL, 0.211 mmol) and (iPr₂N)₂PO(CH₂)₂CN (121 μL, 0.384 mmol) were dropped, and stirred at room temperature for 1 hour. The reaction was confirmed by TLC (CHCl₃:MeOH=9:1), and stirring was stopped. The reaction solution was transferred to an analytical funnel and washed several times with NaClaq. The organic phase was then dried with Na₂SO₄, and the solvent was removed by an evaporator to obtain Compound 6 (95.1 mg, 0.112 mmol, 58.3%).

¹H-NMR (400 MHz, DMSO-d₆) δ SALDI-MS: Calc'd for C₅₁H₅₄N₃NaO₈P [M+Na]⁺=890.3541, Found 890.3544.

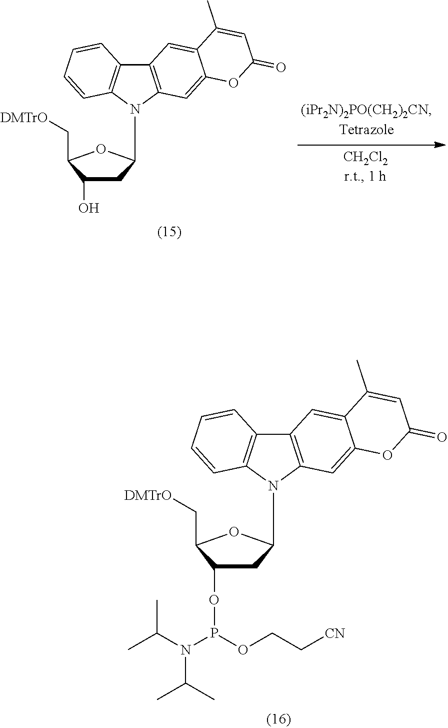

### [Synthesis of Oligonucleic Acid Containing ^{MEP}K]

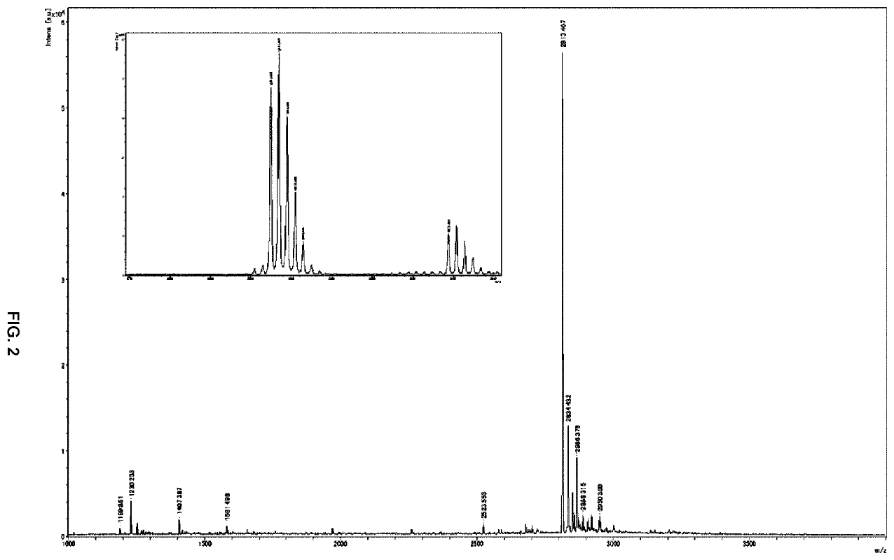

The following sequence (5′-TGCAXCCGT-3′, X=^{MEP}K) was synthesized using an oligo synthesizer. After completion of the reaction, processing was carried out for 30 min using 28% aqueous ammonia (1 mL) (twice), and deprotection was then carried out at 65° C. for 4 hours. Subsequently, the solvent was distilled off by SpeedVack, and the resulting product was dissolved in 100 μL of purified water and purified by HPLC. After that, analysis by MALDI-TOF-MS was carried out to identify a target product. FIG. 2 shows the MS spectrum of the oligonucleic acid containing ^{MEP}K.

Calc'd for [M+H]⁺=2812.528, Found 2813.467.

### [Study of Photocrosslinking of Oligonucleic Acid Containing ^{MEP}K]

### [Photocrosslinking Reaction]

A 50 mM cacodylic acid buffer (pH 7.4) containing 100 μM of ODN1 (5′-TGCAXCCGT-3′, X=^{MEP}K), 100 μM of ODN2 (5′-ACGGGTGCA-3), 50 μM deoxyuridine, and 100 mM of NaCl was annealed, and allowed to stand at 4° C. Subsequently, the resulting product was irradiated with light at 400 nm at 4° C. for 60 seconds using a UV-LED (OmniCure, LX 405-S).

### [HPLC Analysis]

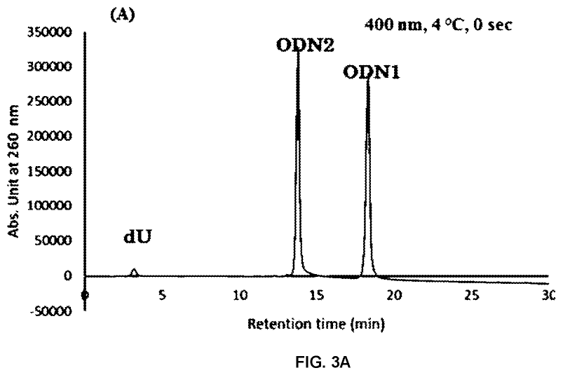

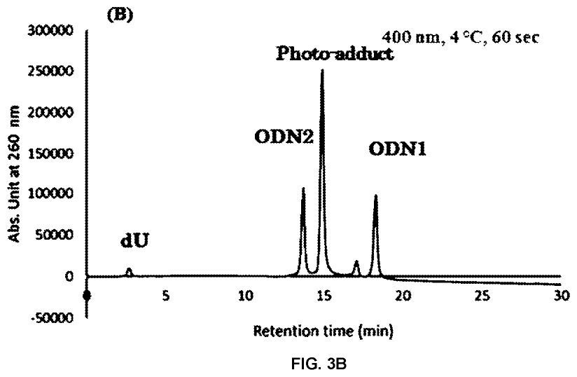

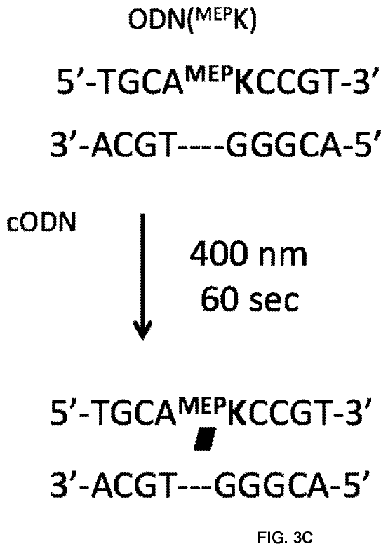

50 μL of a crosslinked sample was analyzed by HPLC. For the analysis, 50 mM of ammonium formate and acetonitrile were used, and a ratio of the solvents was linearly changed such that ammonium formate was 98% at the start of the analysis, and ammonium formate was 70% and acetonitrile was 30% at 30 minutes. The analysis was carried out under conditions of a flow rate of 1.0 mL/min, a column temperature of 60° C., and a detection wavelength of 260 nm. HPLC chromatograms thus obtained before and after irradiation with light are shown in FIGS. 3A and 3B. FIG. 3A is a chromatogram of the crosslinked sample at 0 sec of irradiation with light. FIG. 3B is a chromatogram of the crosslinked sample at 60 sec of irradiation with light. FIG. 3C shows an explanatory view of the photocrosslinking reaction.

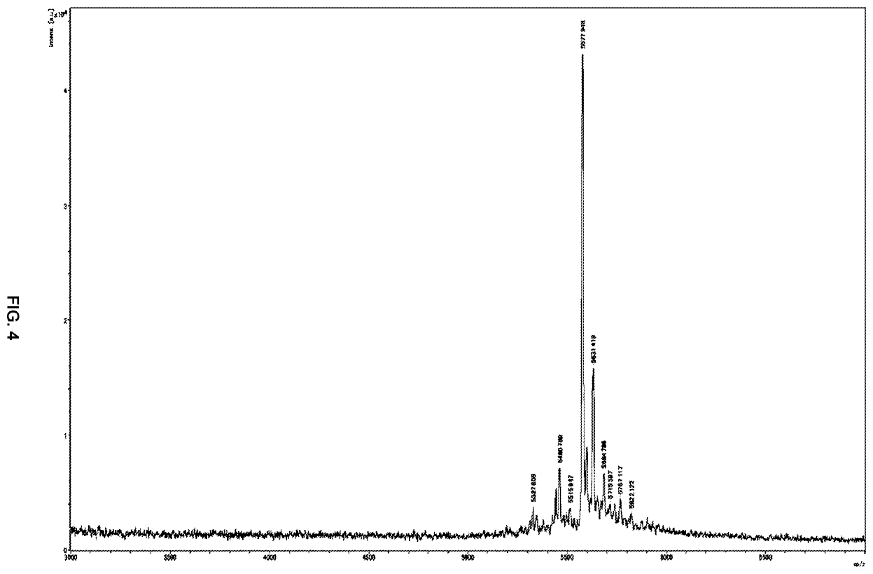

As shown in FIG. 3B, a new peak appeared at a retention time of 15 minutes after 60 seconds of irradiation with light. This peak was fractionated and analyzed by MALDI-TOF-MS to identify a target product. FIG. 4 shows the MS spectrum of the target product (photocrosslinked product).

Calc'd for [M+H]⁺=5575.036, Found=5577.948.

### [Synthesis of Nucleoside Analog (^{MEP}D)]

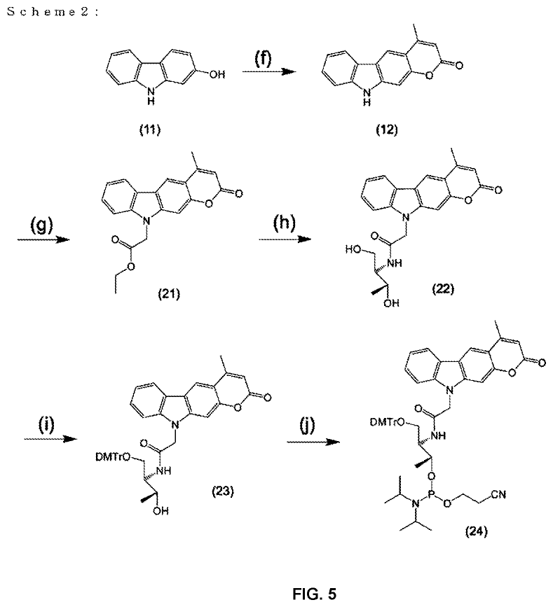

A photoresponsive artificial nucleoside analog molecule (^{MEP}D) was synthesized along the synthetic route as shown in Scheme 2 of FIG. 5, and a modified nucleic acid synthetic monomer was synthesized. Furthermore, a modified DNA into which the monomer was introduced was synthesized. Details for each step will be described later.

In each synthesis step of Scheme 2, the conditions (f) to (j) are as follows. The symbol “r.t.” means room temperature.

(f) Ethyl acetoacetate, H₂SO₄, EtOH, 90° C., 2 h;
(g) NaH, NaI, Ethyl bromoacetate, DMF, r.t., 8 h;

### (h) [1]. NaOH, THF/MeOH/H₂O, r.t., 5 h,

[2]. D-Threoninol, EDCl, HOBt, DMF, r.t., 24 h;

### (i) DMTrCl, DMAP, Pyridine, r.t., 24 h; and

(j) (iPr₂N)₂PO(CH₂)₂CN, tetrazole, CH₃CN, r.t., 4 h.

### [Synthesis of Compound 21]

Compound 12 (300 mg, 1.20 mmol), NaI (540 mg, 3.60 mmol) and NaH (148 mg, 3.60 mmol) were placed in an eggplant flask placed on ice, vacuumed, and then purged with N₂. 10 mL of DMF was slowly added dropwise thereto. After stirring for 20 minutes, ethyl bromoacetate (266 μL, 2.40 mmol) was added, and stirred at room temperature for 8 hours. Disappearance of the raw materials was confirmed by TLC (CHCl₃:MeOH=9:1). After terminating the reaction by adding a small amount of MeOH, the solvent was removed by an evaporator. After removing the solvent, AcOEt was added and the liquid was separated. The organic phase was dehydrated with Na₂SO₄, and the solvent was then removed by an evaporator. After removal, purification was carried out by column chromatography (CHCl₃). The separated compound was dried to obtain Compound 21 (230 mg, 0.688 mmol, 77%).

¹H-NMR (400 MHz, DMSO-d₆) δ 8.59 (s, 1H), 8.28 (d, 1H, 7.68 Hz), 7.61 (s, 1H), 7.57 (d, 1H, 8.16 Hz), 7.49 (t, 1H, J=7.60 Hz), 7.30 (t, 1H, J=7.62 Hz) 6.28 (s, 1H), 5.39 (s, 2H), 4.17-4.14 (m, 2H), 2.58 (s, 3H), 1.22 (t, 3H, 6.12 Hz) ESI-FT-ICR MS: Calc'd for C20H₁₈NO₄ [M+H]⁺=336.1230, Found 336.1230.

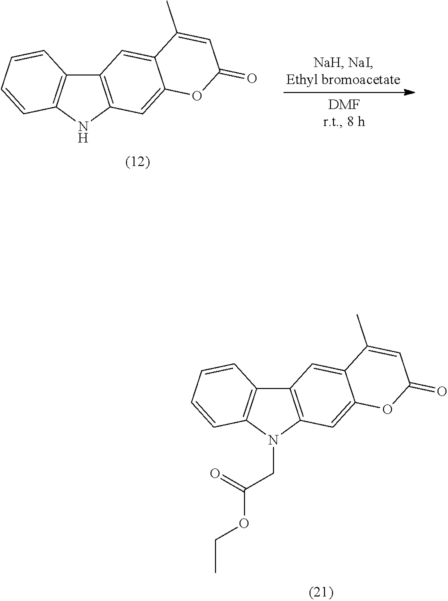 [SMILES: Cc1cc(=O)oc2cc3[nH]c4ccccc4c3cc12 ; CCOC(=O)Cn1c2ccccc2c2cc3c(C)cc(=O)oc3cc21]

### [Synthesis of Compound 22]

Compound 21 (230 mg, 0.688 mmol) and a mixed solvent of THF (9 mL)/MeOH (6 mL)/H₂O (3 mL) were added to an eggplant flask. NaOH (82.6 mg, 1.27 mmol) was added thereto, and the mixture was stirred at room temperature for 5 hours. Disappearance of the raw materials was confirmed by TLC (CHCl₃:MeOH=9:1). HCl aq adjusted to 0.1 M was added to the reaction solution to have a pH of 2. AcOEt was added and the liquid was separated. The organic phase was dehydrated with Na₂SO₄, the solvent was then removed by an evaporator, and the resulting product was vacuum-dried. To the vacuum-dried compound (195 mg) were added DMF (10 mL), D-threoninol (133 mg, 1.37 mmol) and HOBt (172 mg, 1.27 mmol), and the resulting mixture was stirred in N₂ at room temperature for 20 minutes. Subsequently, EDCl (244 mg, 1.27 mmol) was added, and stirred at room temperature for 24 hours. Disappearance of the raw materials was confirmed by TLC (CHCl₃:MeOH=9:1). After terminating the reaction by adding a small amount of MeOH, the solvent was removed by an evaporator. After removing the solvent, AcOEt was added and the liquid was separated. The organic phase was dehydrated with Na₂SO₄, and the solvent was then removed by an evaporator. The resulting product was vacuum-dried to obtain Compound 22 (140 mg, 0.355 mmol, 52%).

¹H-NMR (400 MHz, DMSO-d₆) δ 8.62 (s, 1H), 8.28 (d, 1H, 7.72 Hz), 7.98 (d, 1H, 8.84 Hz), 7.57-7.58 (m, 2H), 7.49 (t, 1H, J=7.64 Hz), 7.29 (t, 1H, J=7.34 Hz) 5.16 (d, 2H, 3.76 Hz), 4.72 (d, 1H, 4.64 Hz), 4.64 (t, 1H, 5.52 Hz), 3.93-3.89 (m, 1H), 3.67-3.62 (m, 1H), 3.54-3.48 (m, 1H), 3.41-3.36 (m, 1H), 2.61 (s, 3H), 1.03 (d, 3H, 6.40 Hz) SALDI-FT-ICR MS: Calc'd for C₂₂H₂₂N₂NaO₅ [M+H]⁺=417.1409, Found 417.1418.

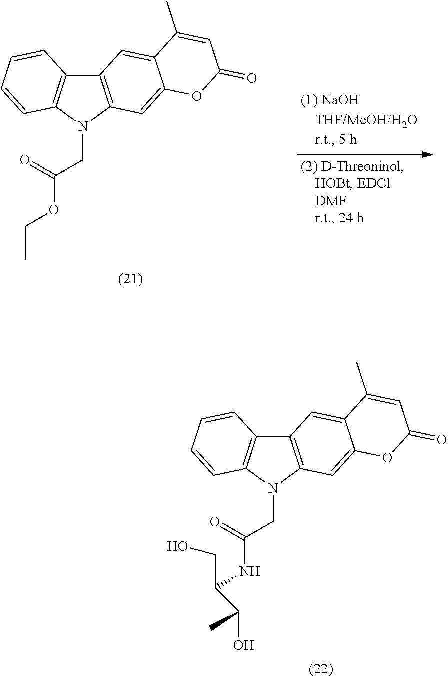

### [Synthesis of Nucleoside Analog (^{MEP}A)]

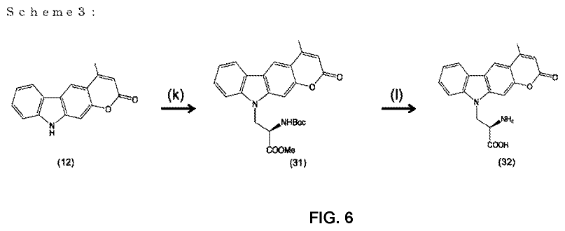

A photoresponsive artificial nucleoside analog molecule (^{MEP}A) was synthesized along the synthetic route as shown in Scheme 3 of FIG. 6. Further, a modified nucleic acid synthesis monomer was synthesized, and a modified DNA into which the monomer was introduced was synthesized. Details for each step will be described later.

### [Synthesis of Boc]

Compound 12 (methylpyranocarbazole) (300 mg, 1.20 mmol), NaI (540 mg, 3.60 mmol) and NaH (148 mg, 3.60 mmol) were placed in an eggplant flask on ice, and the mixture was vacuumed and then purged with nitrogen. 10 mL of DMF was slowly added dropwise thereto. After stirring for 20 minutes, Boc-Ser-OMe bromide (677 mg, 2.40 mmol) was added, and stirred at room temperature for 6 hours. Disappearance of the raw materials was confirmed by TLC (CHCl₃:MeOH=9:1). After terminating the reaction by adding a small amount of MeOH, the solvent was removed by an evaporator. After removing the solvent, AcOEt was added and the liquid was separated. The organic phase was dehydrated with Na₂SO₄, the solvent was then removed by an evaporator. After removal, purification was carried out by column chromatography (CHCl₃). The separated compound was dried to obtain Compound 31. Mass spectrometry was carried out to obtain a target compound (190 mg, 0.422 mol) with a yield of 35%.

FT-ICR MS: Calcd [M+H]⁺: 449.2071, Found 449.2075

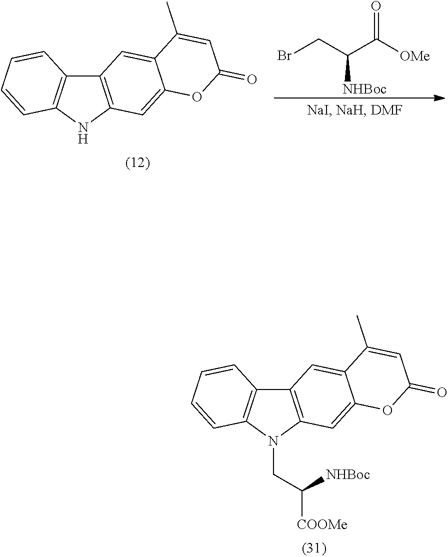

### [Synthesis of ^{MEP}A]

The Boc (206 mg, 0.46 mmol) of Compound 31 (methylpyranocarbazole) and NaOH (180 mg, 23 mmol) were dissolved in THF/MeOH/H₂O (3:2:1, 30 mL) and stirred at room temperature for 2 hours. Subsequently, 1N HCl (250 mL) was added, extraction was carried out with EthOH, and the solvent was then removed by an evaporator. The resulting product was then dissolved in dichloromethane (10 mL), trifluoroacetic acid (3 mL) was added, and the mixture was stirred at room temperature for 12 hours. After TLC (CHCl₃:MeOH=9:1), the resulting product was stained with ninhydrin to confirm a spot. The solvent was removed by an evaporator, and Compound 32 was identified by mass spectrometry.

FT-ICR MS Calcd [M+1-1]⁺=391.0899 found [M+H]⁺=391.0900

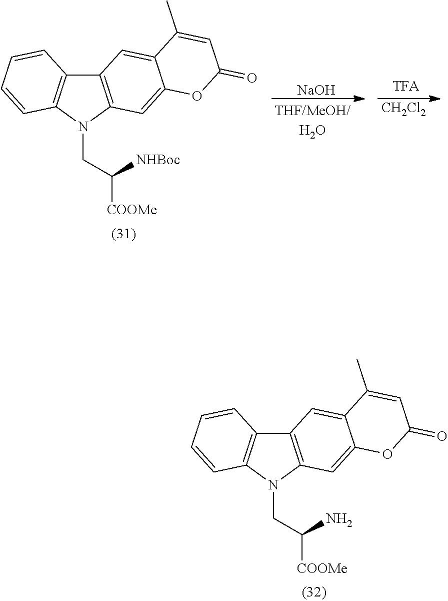

### [Synthesis of Nucleoside Analog (^{PC}X)]

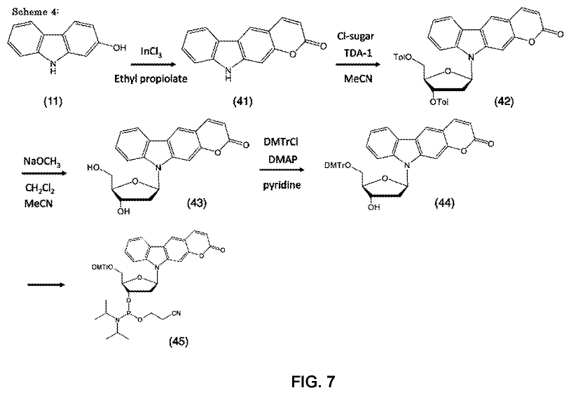

For comparison with the synthesis of the nucleoside analog (MEPK) according to the present invention, synthesis of a nucleoside analog (^{PC}X) was carried out as a comparative example. A photoresponsive artificial nucleoside analog molecule (^{PC}X) was synthesized along the synthetic route as shown in Scheme 4 of FIG. 7, a modified nucleic acid synthetic monomer was further synthesized, and a modified DNA into which the monomer was introduced was synthesized. The synthesis was carried out in the following procedure:

To 2-hydroxycarbazole as a starting material was added InCl₂, and purged with nitrogen. Ethyl propiolate was then added, and stirred at 80° C. for 24 hours or more. Subsequently, the resulting product was purified by column chromatography. Pyranocarbazole was coupled with chlorosugar in acetonitrile, and the trityl group was removed with sodium methoxide in methanol. A nucleoside compound was obtained, which was then tritylated and amidated according to a conventional method to obtain Compound 45.

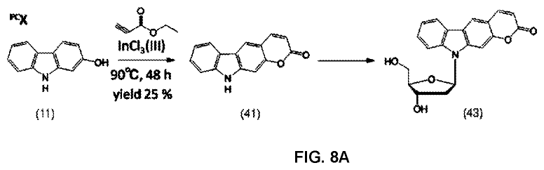

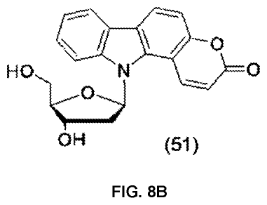

FIG. 8A shows a schematic explanatory view of the procedure for synthesizing the nucleoside analog (^{PC}X). The rightmost product in FIG. 8A is the nucleoside analog (^{PC}X). The nucleoside analog (^{PC}X) has the same structure as that of the nucleoside analog (^{MEP}K) except for the presence or absence of the methyl group, that is, they also have the pyranocarbazole skeleton. The present inventors have found that the nucleoside analog (^{PC}X) can be synthesized by the procedure in FIG. 8A. However, in the synthesis by the procedure in FIG. 8A, the isomer as shown in FIG. 8B was generated, so that the yield was lower and the cost was increased.

[Comparison of Nucleoside Analog (^{PC}X) Synthesis with Nucleoside Analog (^{MEP}K) Synthesis]

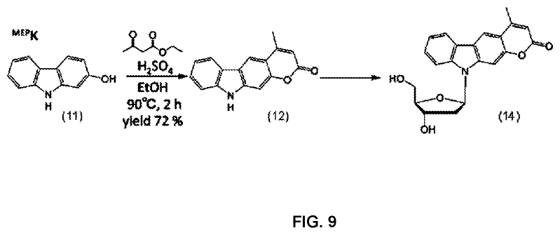

FIG. 9 shows an explanatory view of the outline of the procedure for synthesizing the nucleoside analog (^{PC}X) described above in Examples of the present application, which is summarized so as to be easily compared with FIG. 8A. When compared with the synthesis by the route in FIG. 8A, the synthesis by the route in FIG. 9 reduced the synthesis cost per mol from the compound at the left end in each figure to the compound at the center in each figure to 1/100 times for the cost of the condensing agent, 1/3000 times for the cost of the acid catalyst, and shortened the synthesis time to 1/24 times, and significantly improved the yield from 25% to 72%. That is, in synthesizing a compound having a similar structure except for the presence or absence of a methyl group, the synthesis method according to the present invention significantly improved the cost, time, and yield as compared with the conventional methods.

[Comparison of ^{PC}X Synthesis Procedure with ^{MEP}K, ^{MEP}D, ^{MEP}A Synthesis Procedures]

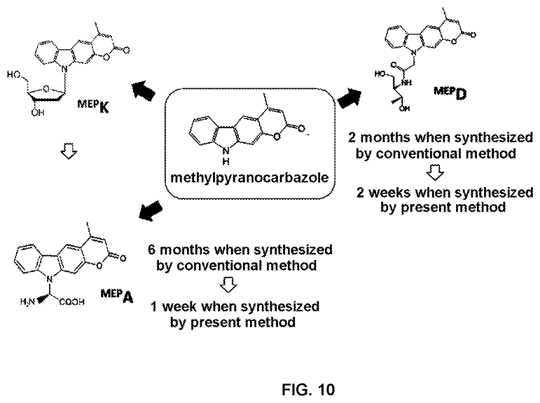

As described above in Examples, all of the procedures for synthesizing ^{MEP}K, ^{MEP}D, and ^{MEP}A have a common step of synthesizing the compound at the right end of FIG. 9 into the compound at the center. As shown in the procedure for synthesizing ^{PC}X, this step can be carried out by the step of synthesizing the compound at the right end to the compound at the center in FIG. 7A to synthesize a compound having the same structure except for the presence or absence of a methyl group. Therefore, when comparing the times required for the synthesis of the compounds having the same structure except for the presence or absence of the methyl group thus obtained, the time for ^{MEP}K was shortened from one month to one week, and the time for ^{MEP}D was shortened from 2 months to 2 weeks, and the time for ^{MEP}A was shortened from 6 months to 1 week. The reason why these shortened times are not uniform is that the compounds shown in FIG. 8B and other compounds are produced as by-products, and the by-products produced by the subsequent operations are further diversely increased, and as a result, removal operations are required for the respective by-products. FIG. 10 shows an explanatory view of the comparison results.

### INDUSTRIAL APPLICABILITY

According to the present invention, a compound as a photoreactive crosslinker that can be used in a photoreaction technique of a nucleic acid can be produced in a short period of time with higher yield. The present invention is an industrially useful invention.

1.-6. (canceled)

7. A photoreactive crosslinking agent comprising the compound of the following formula I: 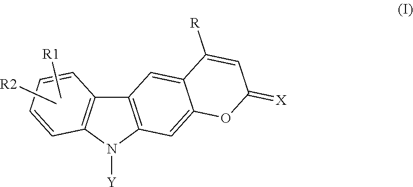
in which formula I:
R is a Methyl group;
X is an oxygen atom;
R1 and R2 are each independently a group selected from the group consisting of a hydrogen atom, a halogen atom, a —OH group, an amino group, a nitro group, a methyl group, a methyl fluoride group, an ethyl group, an ethyl fluoride group, and a C1-C3 alkylsulfanyl group; and
Y represents a saccharide including ribose and deoxyribose; a polysaccharide including a polyribose chain and a polydeoxyribose chain of a nucleic acid; a polyether; a polyol; an alkanolamine; an amino acid; a polypeptide chain including a polypeptide chain of a peptide nucleic acid; or a water-soluble synthetic polymer.

8. The photoreactive crosslinking agent according to claim 7, wherein the group Y in the formula I is a group selected from the group consisting of atoms and groups represented by the following (i) to (iv):
(i) a group represented by the following formula Ya: 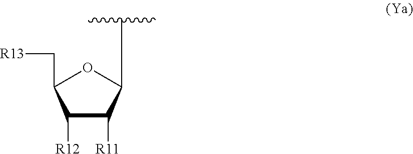
in which formula Ya:
R11 is a hydrogen atom or a hydroxyl group,
R12 is a hydroxyl group or a —O-Q₁ group,
R13 is a hydroxyl group or a —O-Q2 group,
Q₁ is a group selected from the group consisting of:
a phosphate group formed together with O bonded to Q₁;
a nucleotide, nucleic acid or peptide nucleic acid linked via a phosphodiester bond formed by a phosphate group formed together with O bonded to Q₁; and
a protecting group selected from:
a trityl group, a monomethoxytrityl group, a dimethoxytrityl group, a trimethoxytrityl group, a trimethylsilyl group, a triethylsilyl group, a t-butyldimethylsilyl group, an acetyl group, and a benzoyl group;
Q2 is a group selected from the group consisting of:
a phosphate group formed together with O bonded to Q₂;
a nucleotide, nucleic acid or peptide nucleic acid linked via a phosphodiester bond formed by a phosphate group formed together with O bonded to Q₂; and
a protecting group selected from:
a 2-cyanoethyl-N,N-dialkyl(C1-C4)phosphoramidite group, a methylphosphonamidite group, an ethylphosphonamidite group, an oxazaphospholidine group, a thiophosphite group, a TEA salt of —PH(═O)OH, a DBU salt of —PH(═O)OH, a TEA salt of —PH(═S)OH, and a DBU salt of —PH(═S)OH;
(ii) a group represented by the following formula Yb: 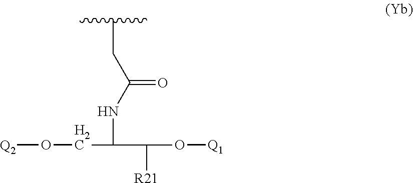
in which formula Yb:
R21 represents a hydrogen atom, a methyl group, or an ethyl group;
Q1 is a group defined as Q1 in the formula Ya; and
Q2 is a group defined as Q2 in the formula Ya; and
(iii) a group represented by the following formula Yc: 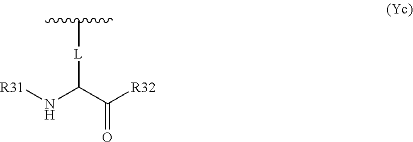
in which formula Yc:
R31 represents a protecting group for the amino group, a hydrogen atom, or a polypeptide linked by a peptide bond formed together with NH bonded to R31;
R32 represents a hydroxyl group, or a polypeptide linked by a peptide bond formed together with CO bonded to R32; and
L is a linker moiety or a single bond.

9. A photoreactive crosslinking agent according to claim 8,
wherein the group Y in the formula I is a group represented by the formula Ya, in which formula Ya:
R11 is a hydrogen atom or a hydroxyl group,
R12 is a hydroxyl group or a —O-Q₁ group,
R13 is a hydroxyl group or a —O-Q₂ group,
Q₁ is a group selected from the group consisting of:
a phosphate group formed together with O bonded to Q₁;
a nucleotide or nucleic acid linked via a phosphodiester bond formed by a phosphate group formed together with O bonded to Q₁; and
Q₂ is a group selected from the group consisting of:
a phosphate group formed together with O bonded to Q₂;
a nucleotide or nucleic acid linked via a phosphodiester bond formed by a phosphate group formed together with O bonded to Q₂;
excepting the case when R1 and R2 are hydroxyl groups at the same time.

10. A method for forming a photocrosslink between nucleic acid bases each having a pyrimidine ring, using the photoreactive crosslinking agent according to claim 7.

11. A method for forming a photocrosslink between nucleic acid bases each having a pyrimidine ring, using the photoreactive crosslinking agent according to claim 8.


SEQUENCE LISTING (2 sequences)

SEQ ID NO: 1 - DNA, 9 bp, Artificial Sequence
  Inventor Fujimoto, Kenzo
  ODN1
  misc_feature (5)..(5): n is 1-methylpyranocarbazole-2-deoxyribose.
tgcanccgt

SEQ ID NO: 2 - DNA, 9 bp, Artificial Sequence
  ODN2
acgggtgca